(19) United States
(12) Patent Application Publication
(10) Pub. No.: US 2011/0178449 A1
(43) Pub. Date: Jul. 21, 2011

(54) TRACTION DEVICE

(76) Inventors: Brian Foote, Blackburn (GB); Patricia Foote, Oswaldtwistle (GB)

(21) Appl. No.: 12/447,275

(22) PCT Filed: Oct. 29, 2007
(86) PCT No.: PCT/GB2007/050659

§ 371 Date: Jan. 24, 2011

(30) Foreign Application Priority Data
Oct. 28, 2006 (GB) 0621499.3

(51) Int. Cl. A61F 5/00

(52) U.S. Cl. 602/36

(57) ABSTRACT

Disclosed is a traction device for applying a tensile force to a first part of a patient's body, the device comprising: an engagement device for securely trapping the first part of a patient's body; and a resilient member arranged to receive the engagement device and to co-operate with a second part of the patient's body to apply the tensile force.

The present invention is concerned with a traction device suitable for use in treating a variety of different fracture types which are likely to be suffered by various bones in the human body. Embodiments of the invention are particularly useful in treating fractures to the wrist such as Scaphoid or Colles fractures.

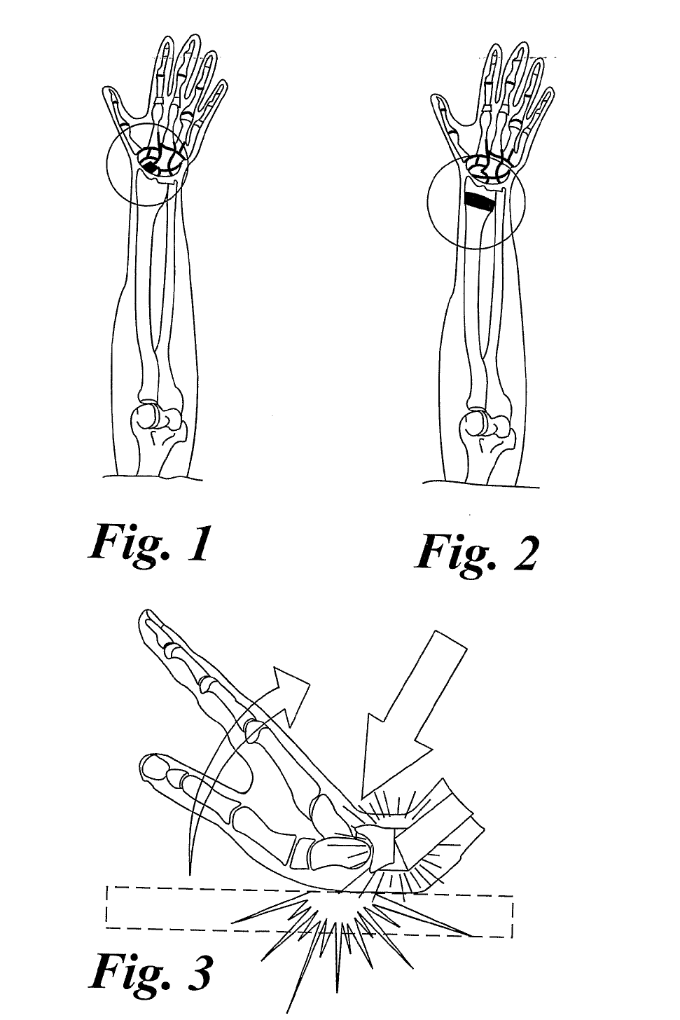

When a person breaks his or her wrist any one or more of a number of bones may be fractured. The most common types of wrist fractures are those involving one of the wrist bones, such as Scaphoid fracture (as shown in FIG. 1), and those involving a break at the end of one of the forearm bones (the Ulna or Radius).

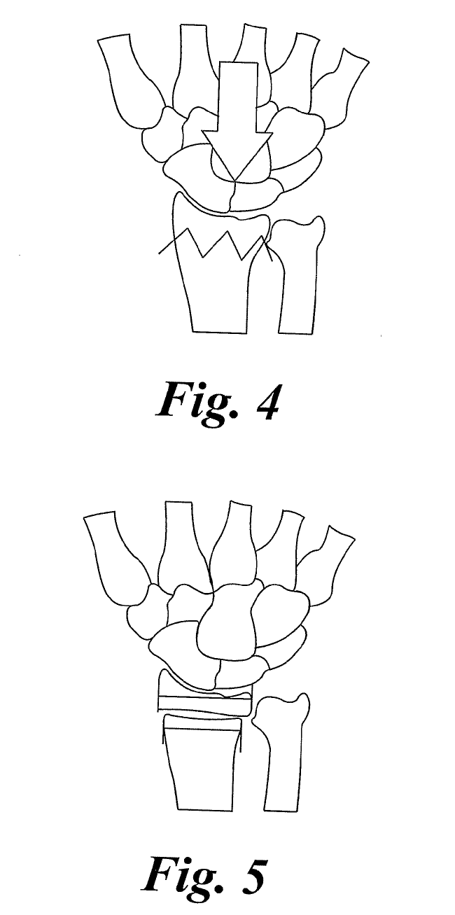

Wrist fractures can occur in a number of different situations but most commonly involve a fall or a motor vehicle accident. FIG. 3 shows a typical impact injury, resulting in a Colles fracture. A Colles fracture, in particular, can be caused by a strong force pushing the hand back into the forearm. This is shown in FIG. 4, with the strong force being represented by the arrow. This is because there is a natural weak spot in the Radius bone at its end where it widens.

Due to the flare in the shape of the bone at its end, as shown in FIG. 5, the broken piece next to the hand is usually wider than the piece next to it. If the broken pieces of bone do not lock together perfectly, like a jigsaw, the pull of the forearm muscles can act to pull the larger end over the small part like a sleeve. This can result in the bone shortening like a collapsible plastic drinking cup.

Prior art treatment techniques for fractures of this sort can involve manual traction being applied to the patient's fingers, quite often by a medical practitioner simply pulling the fingers so that the broken bones are forced into realignment. This can be very unsatisfactory as it is often difficult to assess whether the bones are correctly aligned before plaster is applied to the fracture. If the bones are not correctly realigned before setting, this can lead to painful joints which may suffer further problems in the future, such as arthritis.

One prior art technique which is used to treat Colles fractures involves the patient being immobilised on a bed, with the affected arm situated over the side of the bed and a system of weights and pulleys being applied to various of the patient's fingers in order to keep the fracture site in traction. This is undesirable as the patient is totally immobilised when only their hand has been injured.

There therefore exists a desire amongst medical practitioners to produce a more practical and patient-friendly solution to the problem of treating Colles and other wrist fractures.

Embodiments of the present invention aim to address this and other problems associated with prior art treatment techniques of Colles fractures and other wrist injuries, such as Carpal Tunnel Syndrome (CTS), whether mentioned herein or not.

According to the present invention there is provided an apparatus as set forth in the appended claims. Preferred features of the invention will be apparent from the dependent claims, and the description which follows.

For a better understanding of the invention, and to show how embodiments of the same may be carried into effect, reference will now be made, by way of example, to the accompanying diagrammatic drawings in which:

FIG. 1 shows a representation of a Scaphoid fracture;

FIG. 2 shows a representation of a Colles fracture;

FIG. 3 shows a representation of a typical impact injury, resulting in a Colles fracture;

FIG. 4 shows a further typical injury, resulting in a Colles fracture;

FIG. 5 shows a close up detailed view of a Colles fracture;

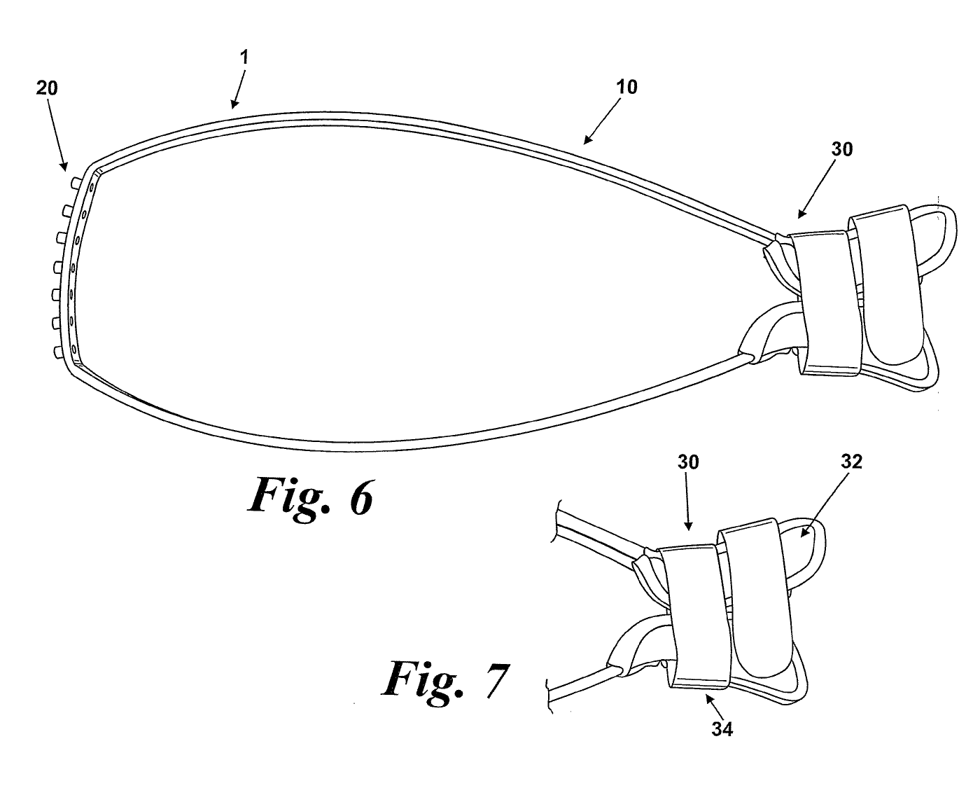

FIG. 6 shows a plan view of an embodiment of the present invention;

FIG. 7 shows a close up view of a cuff portion of the device shown in FIG. 6;

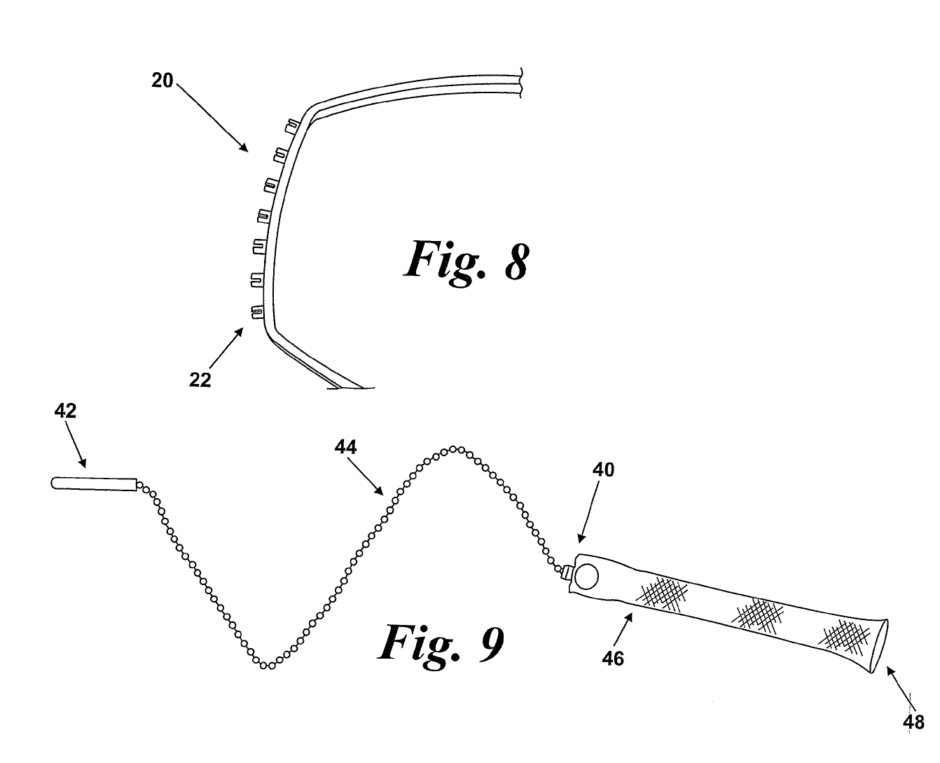

FIG. 8 shows a detailed view of a portion of the device of FIG. 6;

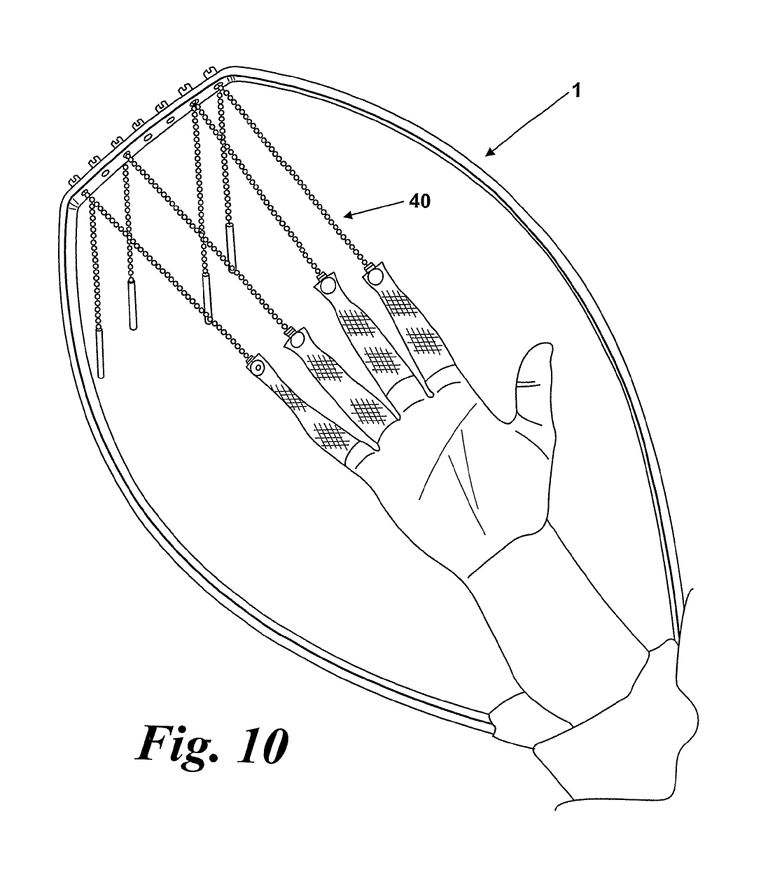

FIG. 9 shows a finger trap and chain for use with the device of FIG. 6; and

FIG. 10 shows an embodiment of the invention in use.

FIG. 6 shows an apparatus according to an embodiment of the present invention. The apparatus 1 comprises a contiguous curved resilient member 10. The apparatus 1 further comprises a cuff portion 30 for engagement with a limb of a patient. Arranged opposite the cuff portion 30 is an attachment area 20 comprising a plurality of individual retaining elements 22 for receiving digit engagement devices 40.

In use, the apparatus 1 is arranged such that the patient's hand is inserted through the cuff portion 30 and securely fastened in position by the use of fastening members 32. FIG. 7 shows a close up view of the cuff portion 30 which comprises a pair of the fastening members composed of hook and eye material such as Velcro® Other fastening means, such as straps and buckles may be provided instead.

Once the patient's hand is inserted into the apparatus, one or more digit engagement devices 40, as shown in FIG. 9, are attached to the patient's digits (fingers or thumb). The digit engagement devices 40 comprise a finger trap element 46 which has a slightly flared end 48. The finger trap element 46 comprises a woven mesh like material which when compressed in a longitudinal direction allows easy insertion of a patient's digit and when extended in a longitudinal direction, the cross sectional diameter of the element 46 is reduced, trapping the finger or thumb inside the device 46.

The finger trap element may be provided in a range of different sizes to suit different digits and, indeed, different patients.

Once one or more digit engagement devices 40 have been applied to a patient's digits, as required, tension may be applied to the digits by use of the chain 44 attached to an opposite end of the device to the flared portion 48.

At the end of the chain, is a small rigid longitudinal element 42 arranged for insertion through element 22 located at the end of the apparatus remote from the cuff portion 30.

The chain 44 comprises a plurality of links formed from individual, generally spherical, portions linked by short longitudinal wires. This type of chain is commonly seen attached to bath plugs and the like.

The entire digit engagement device 40 may be composed of materials suitable to be sterilised by e.g. autoclave or, more preferably, may be intended for single use and subsequent disposal.

Once the insertion portion 42 has been passed through retaining element 22, the requisite tension can be applied to the digit inserted into the digit engagement device 44 and the chain can be locked into that position simply by turning it and anchoring it in the slot portion formed in element 22. This may be repeated for as many digits as are to be held in traction in this way.

The resilient element 10 is generally composed of a suitable plastics material, such as polycarbonate. This has the required resilience to flex under tension but not to shatter or otherwise break. In effect, element 10 acts as a large elliptical spring which keeps the digits in tension. Other materials, including other plastics material or metals may be used.

FIG. 10 shows a view of the apparatus 1 in use with a patient's hand inserted therein and four digit engagement devices 40 attached to the patient's fingers and kept in tension. In this way, suitable tension or force may be applied to the joints of the fingers and hence to the wrist allowing the wrist fracture to be suitably positioned for a plaster cast or other treatment to be applied.

Embodiments of the invention may also find use in surgery on the hand or wrist, where they may be used as a wrist distractor to hold at least part of the wrist in traction, allowing certain types of surgery to be performed more easily.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A traction device for applying a tensile force to a first part of a patient's body, the device comprising:
an engagement device for securely trapping the first part of a patient's body; and
a resilient member arranged to receive the engagement device and to co-operate with a second part of the patient's body to apply the tensile force.

2. The traction device of claim 1 wherein the engagement device comprises a webbed portion for surrounding the first part of the patient's body.

3. The traction device of claim 1 wherein the engagement device comprises an elongate element for co-operation with the resilient member.

4. The traction device of claim 3 wherein the elongate element comprises a length of metallic chain.

5. The traction device as claimed in claim 3 wherein the elongate element is terminated at its free end with a substantially rigid guide-piece.

6. The traction device according to claim 1 wherein the resilient member is composed of one of: metallic substance; a plastics material; and a wooden material.

7. The traction device of claim 6 wherein the resilient member is composed of polycarbonate.

8. The traction device of claim 3 wherein the resilient member comprises a hollow tube member which is provided with a slot along its length so that the elongate member can be retained at a selected tension.

9. The traction device of claim 1 wherein the resilient member is provided with a cuff portion to surround the second part of the patient's body.

10. The traction device according to claim 1 wherein the resilient member is in the form of a closed loop.

11. A traction device for applying a tensile force to a part of a patient's body, the device comprising:
a resilient member including a pair of spaced-apart curved elements having first and second ends, the first ends interconnected by a transverse element;
a retaining element carried by the transverse element;
a cuff adapted to receive a patient's limb, the cuff coupled to the second ends of the curved elements;
a hollow tube-like finger trap element which is structured so as to constrict in diameter in response to being longitudinally extended; and
an adjustable-length flexible elongated element interconnecting the finger trap element and the retaining element, the elongate element including first and second ends, where the first end of the elongate element is connected to the finger trap element.

12. The traction device of claim 11 wherein the finger trap element comprises a woven mesh material.

13. The traction device of claim 11 wherein the elongated element comprises metallic chain.

14. The traction device of claim 11 wherein the elongated member is terminated at its second end with a substantially rigid longitudinal element.

15. The traction device of claim 11 wherein the resilient member comprises polycarbonate.

16. The traction device of claim 11 wherein the curved elements and the transverse element are integrally formed as a single component.

17. The traction device of claim 11 wherein the retaining element comprises a hollow tube aligned with a corresponding aperture in the transverse element, the tube having a slot formed along its length which is open to a distal end of the tube.

18. The traction device of claim 11 comprising a plurality of finger trap elements coupled to a plurality of retainer elements which are arrayed in a row along the transverse element.
